(19) United States
(12) Patent Application Publication
(10) Pub. No.: US 2015/0068522 A1
(43) Pub. Date: Mar. 12, 2015

(54) NEBULIZER MOUTHPIECE

(71) Applicant: HSINER Co., Ltd., Taichung City (TW)

(72) Inventor: Eric CHANG, Taichung City (TW)

(73) Assignee: HSINER Co., Ltd., Taichung City (TW)

(21) Appl. No.: 14/025,212

(22) Filed: Sep. 12, 2013

(51) Int. Cl. A61M 15/00; A61M 16/08; A61M 16/14

(52) U.S. Cl. CPC A61M 15/0021; A61M 16/14; A61M 16/0875
USPC 128/203.12

(57) ABSTRACT

A nebulizer mouthpiece includes a hollow main body and a connecting tube. The main body is a one-piece element, and includes an annular wall defining an inner space and having a middle section, an outlet section, and a storage section connected respectively to two ends of the middle section. The middle section has an inner diameter not smaller than 30 mm. The connecting tube is adapted for interconnecting the middle section and a liquid container, and surrounding a central axis. A distance between the central axis and a distal end of the storage section is not greater than three times the inner diameter. Liquid received in the liquid container is turned into mist and introduced into the inner space through the connecting tube.

### BACKGROUND OF THE INVENTION

1. Field of the Invention

The invention relates to medical equipment, more particularly to a nebulizer mouthpiece.

2. Description of the Related Art

Referring to FIG. 1, a conventional nebulizer mouthpiece 1 is used with a container 2 and a pipe 3. Liquid-form medicine is stored inside the container 2, and is transported from the container 2 through the conventional nebulizer mouthpiece 1 and into a user's mouth as mist-form by pressured gas released through the pipe 3.

Referring to FIG. 2, the conventional nebulizer mouthpiece 1 includes a T-shaped connecting tube 101, a mouth-receiving piece 102, and a bellows tube 103. The container 2, the mouth-receiving piece 102 and the bellows tube 103 are respectively connected to the three tube sections 105, 106, 104 of the connecting tube 101. The connecting tube 101 and the bellows tube 103 are standard products, and the inner diameter of the section 104 for connecting the bellows tube 103 is 20 millimeters.

Since the bellows tube 103 is corrugated, since the smallest inner diameter of the bellows tube 103 is smaller than that of the sections 104, 106 and since the mouth-receiving piece 102 is tapered away from the section 106, once the connecting tube 101, the mouth-receiving portion 102 and the bellows tube 103 are connected together, the inner annular surface of the conventional nebulizer mouthpiece 1 in a horizontal direction initially goes through a repeated increasing/decreasing change in the bellows tube 103, then increases from the bellows tube 103 to the section 104 and then decreases in mouth-receiving piece 102 thus leading to the following drawbacks.

a) The inner diameter of the section 104 is 20 millimeters, so when the medicine is transported from the container 2 through the section 105 into the conventional nebulizer mouthpiece 1 under high pressure, the mist-form medicine will impact the top side of the section 104 and even condense into liquid form and flow back into the container 2, thereby reducing the amount of mist-form medicine inhaled by a user and affecting its dose and effect.

b) Due to the inconsistently changing inner diameter of the conventional nebulizer mouthpiece 1 in the horizontal direction (i.e. , from small to large to small) not only might the mist-form medicine be trapped and turned into liquid form, but unbalanced and disturbing air flow may also be created.

c) The length of the bellows tube 103 is too long and easily interferes with nearby objects and people, causing discomfort and inconvenience.

### SUMMARY OF THE INVENTION

Therefore, the object of the present invention is to provide a nebular mouthpiece that can eliminate the aforesaid drawbacks of the prior art.

According to the present invention, there is provided a nebulizer mouthpiece including a hollow main body and a connecting tube. The main body is formed into a one-piece element, and includes an annular wall. The annular wall defines an inner space and has a middle section, an outlet section, and a storage section. The outlet section is connected to an end of the middle section. The storage section is connected to another end of the middle section. The middle section has an inner diameter that is not smaller than 30 millimeters.

The connecting tube is adapted for interconnecting the middle section of the main body and a liquid container, and surrounds a central axis. A distance between the central axis and a distal end of the storage section is not greater than three times the inner diameter. Liquid received in the liquid container is turned into mist and introduced into the inner space through the connecting tube.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic view of a conventional nebulizer mouthpiece adapted to be used with a container and a pipe;

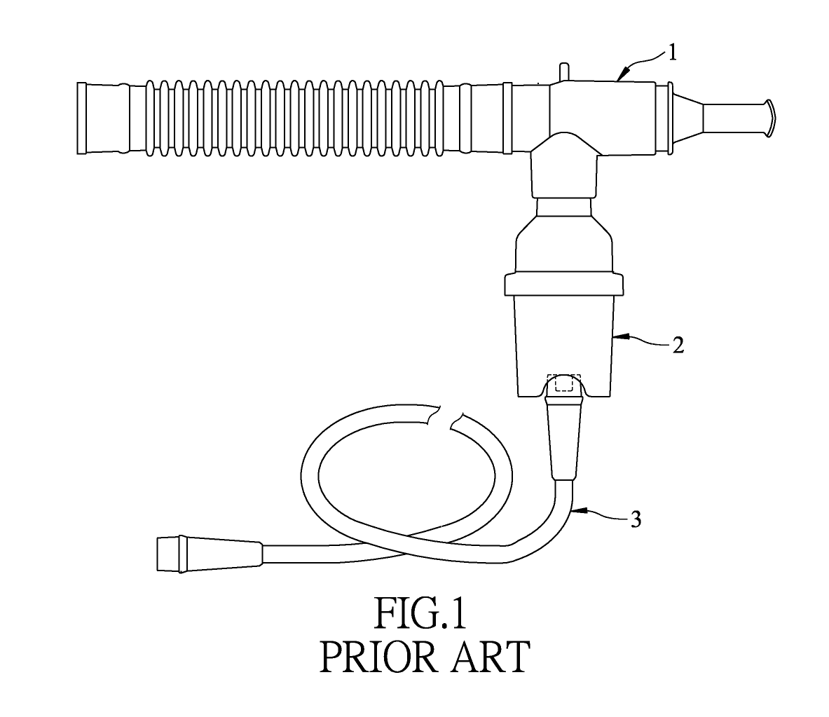

FIG. 2 is an exploded schematic view of the conventional nebulizer mouthpiece;

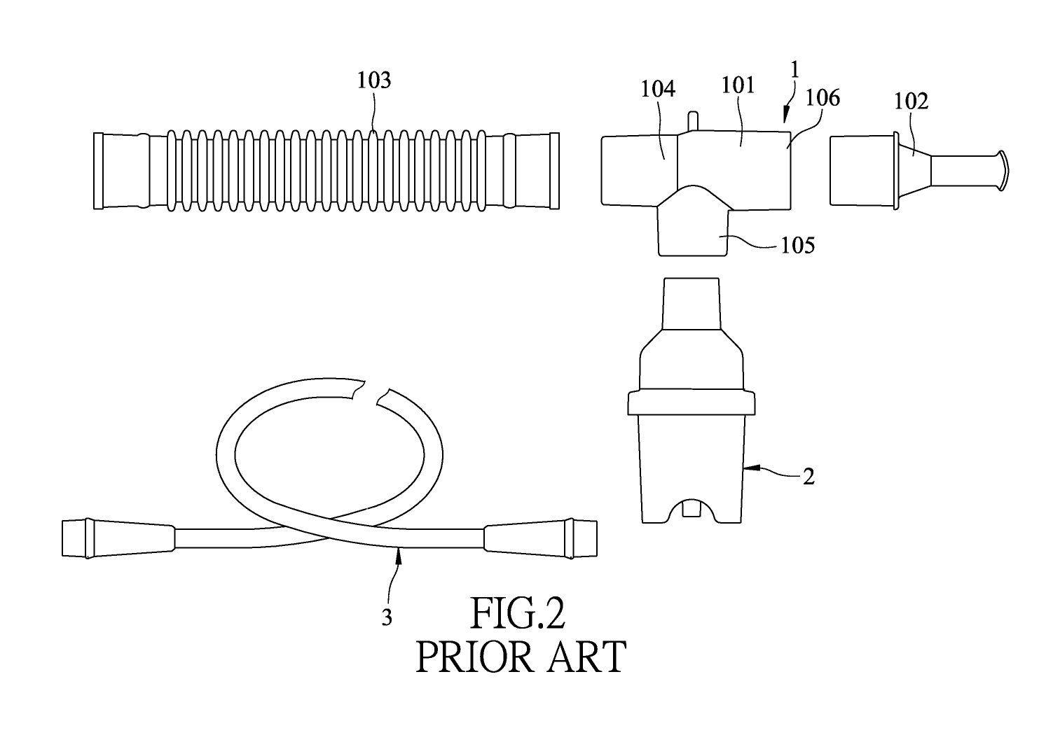

FIG. 3 is an exploded view of an assembly of a preferred embodiment of a nebulizer mouthpiece according to the present invention, a liquid container and a pipe; and

FIG. 4 is a schematic view of the assembly.

### DETAILED DESCRIPTION OF THE PREFERRED EMBODIMENT

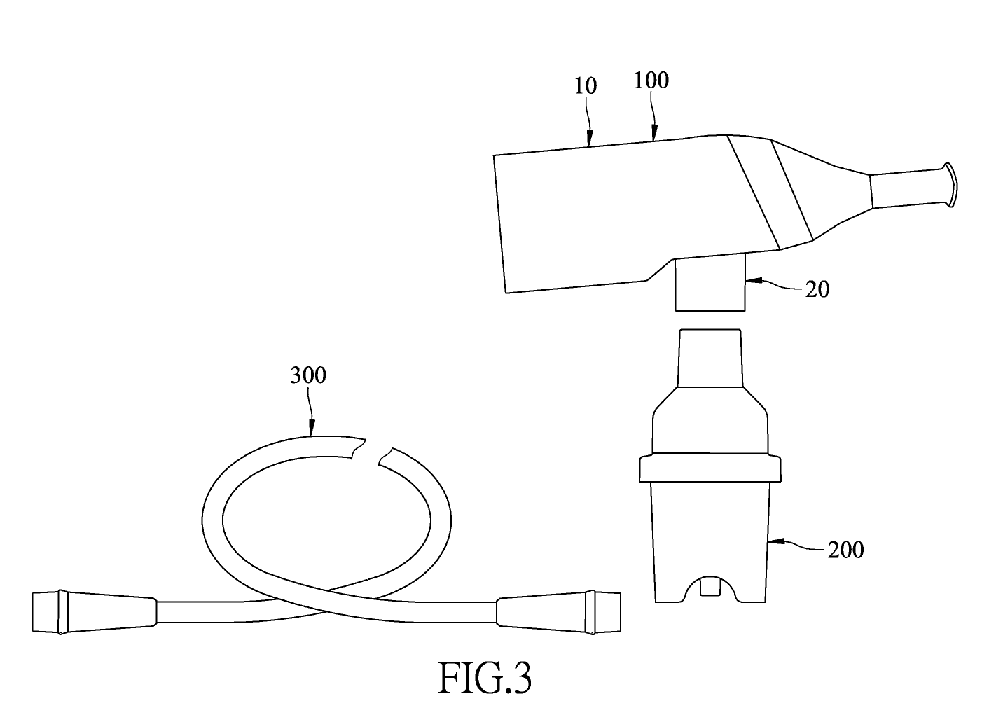

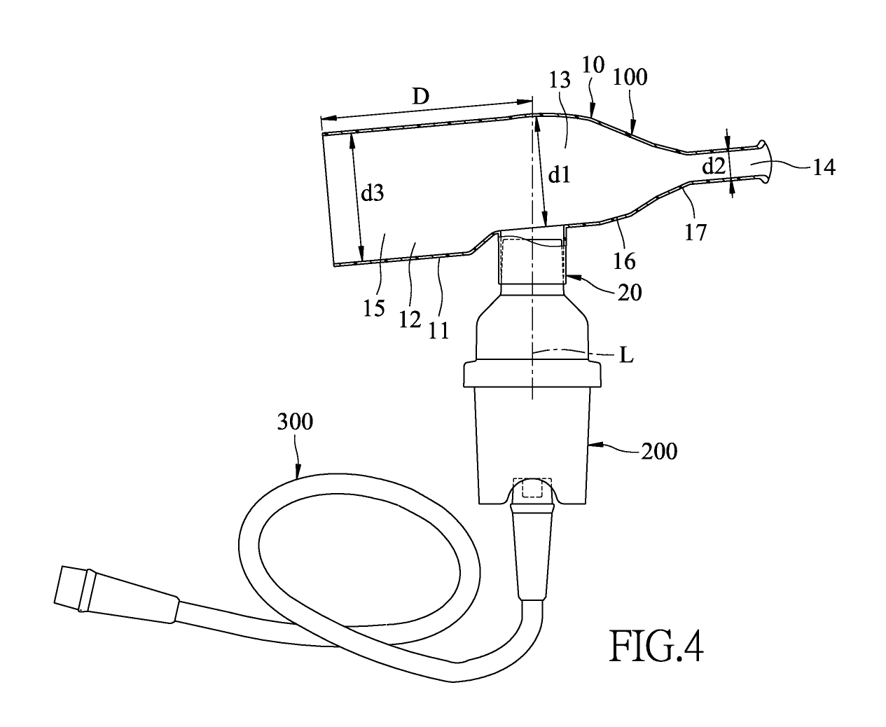

Referring to FIGS. 3 and 4, a preferred embodiment of a nebulizer mouthpiece 100 according to the present invention is adapted to be used with a liquid container 200 and a pipe 300. The nebulizer mouthpiece 100 includes a hollow main body 10 and a connecting tube 20.

The main body 10 is formed into a one-piece element, and includes an annular wall 11. The annular wall 11 defines an inner space 12, and has a middle section 13, an outlet section 14, a storage section 15, a connecting section 16 and a guide section 17. The outlet section 14 is connected to an end of the middle section 13 by the connecting section 16 which is curved in a longitudinal direction of the main body 10, and the guide section 17 which is generally frustoconical and tapers from the middle section 13 toward the outlet section 14. The storage section 15 is connected to another end of the middle section 13. The middle section 13 is aligned with the connecting tube 20, has a smooth inner surface and an inner diameter (d1) that is not smaller than 30 millimeters. The outlet section 14 has an internal diameter (d2) that is smaller than the inner diameter (d1) of the middle section 13. The storage section 15 has a smooth inner surface facing the inner space 12 and an interior diameter (d3) greater than the inner diameter (d1) of the middle section 13.

The connecting tube 20 is adapted for interconnecting the middle section 13 of the main body 10 and the liquid container 200 and surrounds a central axis (L). A distance (D) between the central axis (L) and a distal end of the storage section 15 is not greater than three times the inner diameter (d1) of the middle section 13. Liquid received in the liquid container 200 is turned into mist and introduced into the inner space 12 through the connecting tube 20. In this embodiment, the distance (D) is 62.5 millimeters, and the main body 10 and the connecting tube 20 are integrally formed into one piece. When the liquid container 200 contains liquid-form medicine, the pipe 300 uses pressurized gas to turn the liquid-form medicine into mist-form, which is then transported through the connecting tube 20 into the middle section 13 of the nebulizer mouthpiece 100, and flows toward the connecting and guide sections 16, 17 and out into a user's mouth through the outlet section 14.

The advantages of this invention can be summarized as follows.
a) Since the inner diameter (dl) of the middle section is not smaller than 30 millimeters, when the mist-form medicine is guided into the middle section 13, direct impact against the middle section 13 is reduced due to the greater distance available, preventing the mist form medicine from returning to its liquid form and ensuring the effective amount of the medicine inhaled by the user.
b) Since the inner surfaces of the middle section 13 and the storage section 15 are smooth, and since the transition of inner diameter of the connecting section 16 and the guide section 17 toward the outlet section 14 is also smooth, the flow of the mist-form medicine runs smoother (i.e., not blocked by nor caught between any dead angle created by abrupt depressions or protrusions) into the user's mouth.
c) By ensuring that the distance (D) between the central axis (L) of the connecting tube 20 and the distal end of the storage section 15 to be not greater than three times the inner diameter (dl) of the middle section 13, not only does the storage section 15 still have enough space for the mist-form medicine to move around, but the length of the storage section 15 is also shorter, preventing it from interfering with nearby objects and minimizing discomfort.

It is worth mentioning that even though the main body 10 is a one-piece structure that is simple and easy to manufacture, it allows the mist-form medicine to be guided even more smoothly than the prior art, which enhances the effectiveness of the dose inhaled.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A nebulizer mouthpiece comprising:
a hollow main body formed into a one-piece element, and including an annular wall that defines an inner space and that has a middle section, an outlet section connected to an end of said middle section, and a storage section connected to another end of said middle section, said middle section having an inner diameter that is not smaller than 30 millimeters; and
a connecting tube adapted for interconnecting said middle section of said main body and a liquid container and surrounding a central axis, a distance between the central axis and a distal end of said storage section being not greater than three times the inner diameter;
wherein liquid received in the liquid container is turned into mist and introduced into said inner space through said connecting tube.

2. The nebulizer mouthpiece as claimed in claim 1, wherein said outlet section of said main body has an internal diameter that is smaller than the inner diameter, said storage section having an interior diameter greater than the inner diameter.

3. The nebulizer mouthpiece as claimed in claim 2, wherein said annular wall of said main body further has a guide section interconnecting said middle section and said outlet section, and tapering toward said outlet section.

4. The nebulizer mouthpiece as claimed in claim 1, wherein said main body and said connecting tube are integrally formed into one piece.

5. The nebulizer mouthpiece as claimed in claim 1, wherein said storage section of said main body has a smooth inner surface facing said inner space.
